(19) United States
(12) Patent Application Publication
(10) Pub. No.: US 2022/0076823 A1
(43) Pub. Date: Mar. 10, 2022

(54) COORDINATION UNIT AND TREATMENT SYSTEM

(71) Applicant: Loewenstein Medical Technology S.A., Luxembourg (LU)

(72) Inventor: Peter KREMEIER, Karlsruhe (DE)

(21) Appl. No.: 17/447,219

(22) Filed: Sep. 9, 2021

(30) Foreign Application Priority Data
Sep. 10, 2020 (DE) 102020123601.8

(51) Int. Cl. G16H 40/67; G16H 20/40; G16H 50/80

(52) U.S. Cl. CPC G16H 40/67; G16H 50/80; G16H 20/40

(57) ABSTRACT

A coordination unit for a group of medical devices, which is configured to execute the following steps: receiving sensor data from at least one of the medical devices the sensor data being associated with a patient, receiving a medical data set which is associated with the patient, and initiating an evaluation of the sensor data and/or the at least one medical data set to derive a treatment scheme. Also provided is a treatment system comprising the coordination unit and at least one medical device, the coordination unit being coupled to the medical device (3) via a data connection.

### CROSS-REFERENCE TO RELATED APPLICATIONS

The present application claims priority under 35 U.S.C. § 119 of German Patent Application No. 102020123601.8, filed Sep. 10, 2020, the entire disclosure of which is expressly incorporated by reference herein.

### BACKGROUND OF THE INVENTION

### 1. Field of the Invention

The invention relates to a coordination unit for a group of medical devices and a treatment system comprising the coordination unit. Many modern medical devices, for example, ventilators, permit the request of data via computer networks. The requested data can be used by medical personnel for diagnostic purposes and for documentation purposes.

### 2. Discussion of Background Information

Systems for requesting data from medical devices are known. EP 1571976 B1, the entire disclosure of which is incorporated by reference herein, describes an implantable device which can receive sensor data. The implantable device can be a cardiac pacemaker, for example. Further devices implanted in the body of a patient can transmit sensor data to the implantable device. The implantable device aggregates the data and transmits these data to a remote station outside the body of the patient. EP 2404253 B1, the entire disclosure of which is incorporated by reference herein, describes a medical monitoring system. This can be connected to various medical sensor systems. Upon the occurrence of certain sensor values, the medical monitoring system outputs a warning message. The clinic personnel can thereupon take action.

Therefore, the previously known systems are only capable of aggregating and checking sensor data, but do not assist the treatment of illnesses. In view thereof, it would be advantageous to have available systems which assist a computer-assisted treatment of patients.

### SUMMARY OF THE INVENTION

According to a first aspect thereof, the invention provides a coordination unit for a group of medical devices. The coordination unit is configured to receive sensor data from at least one of the medical devices, wherein the sensor data are associated with a patient, to receive a medical data set which is associated with the patient, and to initiate an evaluation of the sensor data and/or the at least one medical data set to derive a treatment scheme. Therefore, the sensor data and/or the at least one medical data set are not only evaluated, but rather a treatment scheme is also derived therefrom. According to the invention, the treatment scheme can comprise one or more action instructions which can be executed by a person. The treatment scheme can alternatively or additionally comprise one or more action instructions which can be executed by a medical device. Therefore, the coordination unit can initiate required therapeutic measures in reaction to an actual state of a patient. The amounts of data arising in the clinical environment are evaluated and made usable for the treatment of illnesses. It is to be noted that the coordination unit does not or does not at least in all cases derive the treatment scheme independently according to some embodiments of the invention, but rather can also use the processing power of remote computer systems for this purpose.

The coordination unit can optionally be configured to archive patient data which arise during the treatment. The medical data set can contain a data value or a collection of data values which are associated with the patient. The medical data set can thus be, for example, a database entry relating to a patient, an excerpt from an electronic patient file, or at least one value input by a user relating to the patient. Medical personnel can thus, for example, provide missing data values to the coordination unit ad hoc via keyboard input. It is also possible that data from multiple data sources are aggregated in the medical data set, for example, data sets from patient files and archived sensor data.

The coordination unit is preferably furthermore configured to activate at least one of the medical devices so that an implementation of the treatment scheme takes place and/or the implementation of the treatment scheme is assisted. The coordination unit according to the invention can thus transmit action instructions to a medical device which executes the action instructions. For example, the coordination unit can set a mandatory ventilation rate at a ventilator. It is furthermore possible according to the invention that the coordination unit activates medical devices so that the implementation of the treatment scheme is solely assisted. This is the case if not all steps provided according to the treatment scheme can be executed by medical devices. The treatment scheme can provide, for example, that a sedation of the patient is necessary and further steps follow thereon, which can only be performed by medical professionals. In this case, according to one embodiment of the invention, the coordination unit initiates the activation of a syringe pump having a sedative and following steps of the treatment scheme are performed exclusively by the medical professionals.

It is advantageous if the coordination unit is configured to output the treatment scheme, to receive a confirmation signal, and, only upon receiving the confirmation signal, to activate the at least one of the medical devices so that the implementation of the treatment scheme takes place or the implementation of the treatment scheme is assisted. The output of the treatment scheme is to be understood both as a graphic output of the treatment scheme, for example on a display screen, and also a solely technical output of the treatment scheme such as a transmission of the treatment scheme via a computer network. The coordination unit can be configured, for example, to transmit the treatment scheme by means of an ethernet connection or a WLAN connection to a remote station. The coordination unit only activates the at least one of the medical devices if it receives the confirmation signal for this purpose. A user can thus first give feedback to the coordination unit that he has received the treatment scheme and agrees to its implementation before the coordination unit initiates further steps. In this way, it can be ensured that critical therapeutic steps are only initiated by the coordination unit after a check has been carried out by medical personnel.

It is particularly advantageous if the treatment scheme contains a medical diagnosis. The diagnosis can be provided according to the invention by the coordination unit or at least output by the coordination unit. Making the medical diagnosis can include evaluation of data which are available to the coordination unit, in particular of the sensor data and/or the at least one medical data set. According to embodiments of the invention, the coordination unit outputs multiple possible diagnoses. It is possible according to the invention that the treatment scheme couples the medical diagnosis with one or more action options. This enables a user to select one action option and possibly to give the coordination unit an instruction that the action option is to be implemented.

The coordination unit is preferably configured to initiate an output of the treatment scheme. The output is to be understood both as a graphic output of the treatment scheme, for example, on a display screen, and also a solely technical output of the treatment scheme, for example, a transmission via a computer network. It is possible according to the invention that the coordination unit has a display screen to output the treatment scheme. The treatment scheme can thus be visually registered by a user. It is preferred if the coordination unit additionally has an input means. This enables the coordination unit to be able to directly accept requests, confirmations, and other inputs. According to the above statements, the coordination unit thus forms a tool for the unified monitoring and/or control of multiple medical devices.

It is preferred if the input means is a touch display screen. A sufficiently convenient data input may be achieved hereby in the clinical environment. A touch display screen may certainly be cleaned more easily in comparison to a keyboard. The coordination unit is very particularly preferably a tablet computer, thus a portable computer having a touch display screen. However, computer systems of entirely different types, in particular stationary computer systems, can also be used as coordination units.

According to one particular embodiment of the invention, the coordination unit is configured, for the evaluation of the sensor data and/or the at least one medical data set, to place a query to a calculation service and to receive the treatment scheme or data from which the coordination unit can derive the treatment scheme from the calculation service. This is very useful above all if the coordination unit cannot independently execute all steps necessary to derive the treatment scheme for capacity reasons. The calculation service can be implemented by a communication network of distributed computers. Such a communication network is also referred to as a cloud. One advantage of cloud solutions is that a processing power provided by the cloud can be scaled up and down rapidly as a function of the demand.

According to further embodiments of the invention, the calculation service can be implemented by a mainframe computer or a server farm of a hospital. According to the invention, the calculation service can use methods from the field of artificial intelligence, thus, for example, expert systems or neural networks can be used. In this way, treatment schemes may be derived quickly from the sensor data and/or the at least one medical data set. Moreover, a processing load to be borne by the coordination unit can be kept as low as possible.

It is possible according to the invention that the coordination unit is configured to place a database query to a patient database. A patient database is a system which contains patient data in the form of electronic patient files or other database entries relating to patients. For example, key data such as name, date of birth, or residence of a patient can be contained therein, but also directly health-relevant data, for example, items of information on prior illnesses and present illnesses, ordered medications, or presently running treatment measures. These data are preferably provided in the patient database in a standardized format which can be further processed by machine. Medical data sets of this type may be used particularly easily by the coordination unit. The coordination unit can also use the patient database as a data source for the sensor data if these data are stored therein. It is furthermore possible according to the invention that the coordination unit is configured to keep the at least one medical data set ready in an internal database or retrieve it from another remote data source, for example, from a communication network of distributed computers.

The coordination unit preferably requests the sensor data from the medical devices. For this purpose, the coordination unit can be directly connected to the medical devices. However, it is also possible that an indirect data connection exists between the coordination unit and the medical devices. Thus, for example, a coupling device can be provided, to which the medical devices are connected. The coupling device preferably communicates with the medical device via a WLAN connection.

According to one particular embodiment of the invention, the coordination unit is configured to request an availability of the medical devices and to take the availability of the medical devices into consideration in the initiation of the evaluation of the sensor data and/or the at least one medical data set to derive the treatment scheme. It can occur during the treatment of acute illnesses that medical devices are already occupied. In such cases, it is necessary to find alternative treatment options within a short time. Furthermore, medical devices which have a tendency to be utilized less strongly are to be used if possible if a usage of other, more strongly requested medical devices can thus be avoided.

In the following, a hypothetical application example is given. A patient is received on an intensive care unit who has tested positively for COVID-19. The patient has acute respiratory distress syndrome (ARDS). To register the extent of the pulmonary disease, a computer tomography of the thorax is to be carried out. This requires the patient to be brought to a computer tomography device via multiple hospital corridors. In this case, there is a risk of infection for further patients. After the computer tomography is carried out, the computer tomography device would have to be cleaned. The computer topography device would thus not be available to other patients for a relatively long time. For example, it is conceivable that in the case of a further patient for whom a computer tomography examination is actually urgently to take place, instead a laparotomy is carried out because the computer tomography device is still blocked. This situation is not satisfactory. According to the invention, the coordination unit establishes in the case of the COVID-19 patient that a device for electrical impedance tomography (EIT) is also available. This can be used to register the state of the lungs.

The coordination unit therefore applies the treatment scheme so that an examination of the patient by means of the EIT device is proposed. EIT devices measure electrical conductivities inside the body, which permits inferences about the composition of the body tissue. Such a device can be used alternatively to a computer tomography device in certain cases. The computer tomography device is therefore not blocked over a longer time period. In the case of the derivation of the treatment scheme in consideration of the availability of the medical devices, according to advantageous embodiments, the coordination unit can additionally take into consideration a risk of infection existing due to a disease of the patient, a spatial distance of the patient from the medical devices, an average occupancy rate of the medical devices, and/or further parameters.

According to a further embodiment of the invention, it can be provided that the sensor data and/or the medical data set comprise measured values from a device for electrical impedance tomography, a device for blood gas analysis, and/or a ventilator, wherein the medical data set comprises at least one data value relating to a positioning of the patient, and wherein the treatment scheme comprises an instruction for the positioning of the patient derived from the sensor data and/or from the at least one medical data set. According to this embodiment of the invention, the coordination unit is used for assistance in the positioning therapy. This preferably takes place during the treatment of patients who are sick with COVID-19 and receive a ventilation therapy via a ventilator. Measured values from a device for electrical impedance tomography, a device for blood gas analysis, and/or a ventilator are preferably evaluated by the coordination unit. However, further measured values can also be taken into consideration. A suitable positioning therapy may be derived therefrom. However, further clinical parameters can also be evaluated according to the invention. The coordination unit preferably outputs instructions for positioning to the clinical personnel, for example, via a display screen of the coordination unit.

According to a further embodiment of the invention, the sensor data and/or the medical data set comprise at least one coagulation blood value and an oxygenation value, wherein the treatment scheme comprises, derived from the sensor data and optionally from the at least one medical data set, a presence of microthrombi in a lung of the patient as a diagnosis and wherein the treatment scheme provides an administration of an anticoagulant to the patient derived from the sensor data and/or the at least one medical data set. According to this variant of the invention, the coordination unit is configured so that it can diagnose a presence of microthrombi.

For example, if a proportion of D-dimers in the blood increases strongly and the oxygenation worsens, this indicates worsened blood coagulation. The treatment scheme generated by the coordination unit comprises the administration of the anticoagulant to dissolve the microthrombi. For example, a treatment proposal can be given to medical personnel that an anticoagulant is to be administered, or the coordination unit can trigger the administration of the anticoagulant itself, for example by an activation of a syringe pump.

One particular area of application of this embodiment of the invention is the treatment of patients who are sick with COVID-19, because according to current findings, increased blood coagulation accompanies this illness, which can result in thrombi and lung emboli. The coordination unit can initiate the required prophylactic steps in such cases. It is obvious that the administration of the anticoagulant can also be initiated in the case of other diseases by the coordination unit, insofar as this is medically indicated.

The sensor data and/or the medical data set preferably furthermore comprise measured values from a device for electrical impedance tomography, a device for blood gas analysis, and/or a ventilator. The medical data set optionally comprises at least one data value on a positioning of the patient. Examination results from an EIT device can be used to establish the microthrombi. It is possible by means of EIT devices, for example, to prepare a fine-resolution image of the thorax. It is possible according to the invention that the coordination unit is configured to automatically evaluate an image of the thorax so that microthrombi can be recognized. A diagnosis can be derived on this basis in particular in consideration together with further measured values and medical data sets. Alternatively or additionally, at least one measured value which is output by a ventilator can also be evaluated, because respiratory parameters can be influenced by the beginning and progressing occurrence of microthrombi. Finally, a data value on a positioning of the patient can additionally be evaluated. For example, it can be determined how often the positioning position was changed and whether the patient has possibly temporarily left the hospital bed. A more precise statement on the probability of a thrombus formation may be made on this basis.

In each of the above-described embodiments of the invention, it is possible that the medical data set identifies that the patient is infected with a pathogen from the family of the coronaviruses. This can relate in particular to SARS-CoV-2, which is also referred to as severe acute respiratory syndrome coronavirus 2. This causes the illness COVID-19. The coordination unit can be configured according to the invention to take this into consideration in the derivation of the treatment scheme. However, it is obvious that the coordination unit according to the invention can also be used in the case of other diseases.

According to a further advantageous embodiment of the invention, the medical data set records that the patient suffers from muscle weakness acquired on an intensive care unit, wherein the sensor data and/or the medical data set comprise measured values from a device for electrical impedance tomography and/or from a ventilator, wherein the medical data set optionally indicates a sedation status of the patient, and wherein the treatment scheme comprises at least one specific instruction for a withdrawal process from a ventilator derived from the sensor data and optionally from the at least one medical data set. The concept of the muscle weakness acquired on an intensive care unit, also known as ICU-acquired weakness, is to be understood as a severe muscular illness which can be expressed differently. In one possible expression, it is so-called critical illness polyneuropathy, a disease of the peripheral nervous system.

Patients develop slack, atrophic paralyses. This disease can occur in particular as a result of sepsis, multiorgan failure, or long-term ventilation. A further expression of the disease is so-called critical illness myopathy, which can occur in particular in the course of pulmonary diseases. In both expressions of the disease, the withdrawal of the patient from a ventilator can be made significantly more difficult. An ICU-acquired weakness can follow, for example, a COVID-19 disease if a patient has been ventilated on an intensive care unit.

According to the invention, the coordination unit evaluates the above-mentioned data sources to optimally assist the withdrawal from the ventilator. The withdrawal from the ventilator, also called weaning, enables the patient to breathe independently again after a phase of ventilatory assistance. While weaning is generally unproblematic in the case of short ventilation periods, it can prove to be difficult after long-term ventilation. In the specific case, it can take multiple weeks until the patient can breathe completely independently again. With assisted spontaneous respiration by a ventilator, the patient is continuously guided toward independent breathing. A fine adjustment of diverse ventilation parameters can be advantageous.

The coordination unit is preferably configured to perform a corresponding adjustment of the ventilation parameters. The coordination unit particularly preferably activates a ventilator directly. Alternatively or additionally, however, the coordination unit can also output action instructions to medical personnel. A suitable adjustment of ventilation parameters can thereupon be performed manually.

The instruction specific for the withdrawal process is preferably a starting time for the withdrawal process, an ending time for the withdrawal process, or an instruction to adjust ventilation parameters. The starting time for the withdrawal process is to be selected in dependence on a health status of the patient. In general, the weaning can first be begun when diseases have already subsided or healed enough that the patient can manage the withdrawal process. According to the invention, the coordination unit can determine a suitable starting time. For this purpose, in particular the following data sources or parameters can be used according to the invention by the coordination unit: heart rate, blood pressure, inflammation parameters (for example CRP value), body temperature, depth of sedation, blood gas analysis values (e.g., pO2 value, pCO2 value, pH value, hemoglobin level), output data of imaging methods (for example of an MRT device or an EIT device), need for circulatory-support medications (for example retrieved from configuration data of a continuous syringe pump), and/or liquid balance (e.g., supply of infusions, enteral nutrients, and medications in relation to excretions or liquid balance of a dialysis device). Further data sources or parameters can also be used according to the invention to determine the starting time, however.

The coordination unit can similarly propose the ending time for the withdrawal process. In this case, the coordination unit preferably takes into consideration in particular the following data sources or parameters: breathing work (for example determined by a ventilator), RSBI (Rapid Shallow Breathing Index, ratio of the respiratory frequency to the tidal volume), heart rate, blood pressure, inflammation parameters (for example CRP value), body temperature, depth of sedation, blood gas analysis values (e.g., pO2 value, pCO2 value, pH value, hemoglobin level), output data of imaging methods (for example of an MRT device or an EIT device), and/or liquid balance (for example retrieved from configuration data of a continuous syringe pump). Further data sources or parameters can also be used according to the invention to determine the ending time, however.

The instruction for adjusting ventilation parameters can be an instruction according to the invention which is intended to prevent a maladjustment of the ventilator to the patient. It can thus be determined according to the invention whether a negative esophageal pressure exists without pressure decrease by the ventilator in spontaneously breathing patients. This indicates a so-called trigger asynchronism. This means that the ventilator does not respond under spontaneous respiration during inhalation processes and therefore does not assist the patient during the respiration. It can furthermore be determined according to the invention whether a negative esophageal pressure exists before the pressure reduction by the ventilator in spontaneously breathing patients. This can indicate so-called auto-triggering, which means that the ventilator starts although the patient has not yet begun to inhale at all. Both cases mentioned can obstruct the withdrawal process. The coordination unit can accordingly activate a ventilator in a corrective manner and/or output an action proposal for implementation by medical personnel. Corrections can thus be performed which promote success of the weaning.

It is advantageous if the treatment scheme comprises an instruction for setting a ventilation rate, for setting a level of a ventilation pressure, and/or for setting a tidal volume to enable spontaneous breathing. These parameters are preferably to be continuously adjusted in dependence on the course of the weaning. It is particularly advantageous if the coordination unit specifies the treatment scheme so that diaphragm-protective ventilation takes place. There is the possibility of lung damage in the case of machine ventilation. The risk of lung damage can be reduced by suitable adjustment of diverse ventilation parameters, however. Thus, for example, the ventilation pressure, thus the maximum inspirational pressure, is to be kept as low as possible. In the setting of the tidal volume, it is to be taken into consideration that the tidal volume is to be selected to be less the lower the so-called compliance (expansion capacity) of the lungs is. Diaphragm-protective ventilation during the weaning can be carried out in consideration of these and optionally further parameters by means of the coordination unit according to the invention.

According to a further embodiment of the invention, the sensor data originate from a device for electrical impedance tomography, a ventilator, a device for pulse oximetry, a hemoglobin meter, an esophageal pressure sensor, a device for measuring blood pressure, a computer tomography device, an x-ray device, an ultrasound device, a capnometer, an EEG device, and/or a device for blood gas analysis, and the treatment scheme comprises at least one specific instruction for the treatment of an acute respiratory insufficiency of the patient which is derived from the sensor data and/or the medical data set. According to this embodiment, a treatment of an acute respiratory insufficiency of a patient is initiated, in which the above-mentioned sensor data and/or measured values are also incorporated to ensure the best possible treatment.

The at least one specific instruction for the treatment of the acute respiratory insufficiency of the patient is preferably an instruction for setting an inspirational O2 concentration, an instruction for setting an inspiration time, an instruction for setting a PEEP value, an instruction for setting a mandatory ventilation rate, an instruction for setting an individual trigger, and/or an instruction for setting a sedation parameter. In many cases, it can be advantageous if the coordination unit assists the treatment of the acute respiratory insufficiency. An exemplary case is specified hereinafter. A patient requiring ventilation, in whom there is a suspicion of a COVID-19 disease, is received on an intensive care unit. Pronounced respiratory drive, a high need for sedation, and nearly normal compliance of the lungs are present. Imaging diagnostics using, for example, an MRT device are subject to risk due to the unstable pulmonary situation. Lung-protective ventilation to avoid biomechanical complications proves to be very difficult due to end-inspiration overdistention. A safer ventilation window with reduced end-inspiration overdistention can be achieved by setting an individual PEEP value by way of the progressing assessment of the lung situation, the required sedation regime, the blood gases, and the electrical impedance tomography. The abbreviation PEEP refers to the positive end-expiration pressure during the respiration. There is supposed to be a positive pressure during the artificial overpressure ventilation at the end of the expiration to avoid a collapse of the alveoli. A so-called PEEP valve can be used for this purpose.

In all above-described treatment processes, which relate to lung diseases, the coordination unit can be configured according to the invention to derive action instructions from diverse measured values which are incorporated into the treatment scheme. The corresponding treatment knowledge, which can be applied by the coordination unit according to embodiments, is set forth hereinafter. According to one advantageous embodiment of the invention, the coordination unit is configured, in a phase of acute respiratory insufficiency of the patient, to take into consideration parameters comprising a blood pressure, an SpO2 value (oxygen saturation of the blood, for example measured by means of pulse oximetry), a hemoglobin level, and/or a pO2 value. According to the invention, the coordination unit can derive therefrom a required inspirational O2 concentration and/or an advantageous inspiration time. According to a further advantageous embodiment of the invention, the coordination unit is configured, in a phase of acute respiratory insufficiency of the patient, to measure transpulmonary pressures at the end of the exhalation and/or at the end of the inhalation and to derive an individual setting of a PEEP value for a ventilator therefrom. According to a further advantageous embodiment of the invention, the coordination unit is configured, in a phase of acute respiratory insufficiency of the patient, to take into consideration output data of a computer tomography device, an x-ray device, and/or an ultrasound device as an output base for an electrical impedance tomography and to derive a diagnosis of a lung situation of the patient therefrom. The diagnosis can have the result that intensifying the spontaneous respiration is initiated by the coordination unit.

According to a further advantageous embodiment of the invention, the coordination unit is configured, in a phase of acute respiratory insufficiency of the patient, to take into consideration a PEEP value, a pCO2 value, an inspiration pressure, a tidal volume, and/or a result of a volumetric capnometry to set a mandatory ventilation rate of a ventilator. According to a further advantageous embodiment of the invention, the coordination unit is configured, in a phase of acute respiratory insufficiency of the patient, to evaluate a flow curve and/or an esophagus curve in order to set a trigger parameter to trigger a ventilation by a ventilator. The flow curve indicates a respiratory volume over time. The esophagus curve indicates an esophageal pressure over time. The accurate setting of the trigger parameter makes it possible to prevent the respiratory assistance not being triggered or the ventilator triggering a ventilation process although this is not indicated.

According to a further advantageous embodiment of the invention, the coordination unit is configured, in a phase of acute respiratory insufficiency of the patient, to evaluate a setting of a continuous syringe pump, a respiration pattern, a respiration rate, a heart rate, a blood pressure, an esophagus curve, and/or EEG measured values to derive a required depth of sedation. Action instructions to medical personnel to administer required medications for the sedation can be derived therefrom by the coordination unit. According to a further advantageous embodiment of the invention, the coordination unit is configured, in a phase of acute respiratory insufficiency of the patient, to evaluate a setting of a continuous syringe pump for volatile anesthetics, a respiration pattern, a respiration rate, a heart rate, a blood pressure, an esophagus curve, EEG measured values, and/or results of an end-tidal narcosis gas measurement to derive a required depth of sedation. Parameters for setting narcosis gases which are required for sedation can be derived therefrom by the coordination unit.

According to a further advantageous embodiment of the invention, the coordination unit is configured, in a stabilization phase after acute respiratory insufficiency of a patient, to evaluate a ratio of a respiration rate to a tidal volume, a pO2 value, and/or a pCO2 value in order to initiate an adjustment of a frequency of the machine breaths, an adjustment of a level of a ventilation pressure during machine ventilation, and/or an adjustment of a ventilation pressure during machine ventilation. The stabilization phase is to be understood as a time period after acute respiratory insufficiency of a patient in which the weaning has not yet begun. According to a further advantageous embodiment of the invention, the coordination unit is configured, in a stabilization phase after acute respiratory insufficiency of a patient, to determine a pressure delta between a PEEP value and a lowest esophageal pressure during the inspiration in order to determine a measure which reflects whether inadequate spontaneous respiration is present. This measure can be output by the coordination unit as a diagnosis according to the invention.

According to a second aspect, the invention provides a treatment system. The treatment system comprises the above-described coordination unit and at least one medical device, wherein the coordination unit is coupled to the medical device via a data connection. The data connection can be developed basically as desired, but it is preferably to be a wireless data connection such as a WLAN connection in this case. The treatment system can thus receive sensor data of the medical devices and output instructions for implementing the treatment scheme to the medical devices. It is also possible to couple the coordination unit to the medical devices via a wired network. It is also possible that the data connection is established via multiple intermediate stations.

The treatment system preferably has a calculation service, wherein the coordination unit is coupled to the calculation service via a data connection. The calculation service according to the invention can use methods from the field of artificial intelligence. The calculation service according to the invention can thus be configured so that it executes an expert system. The calculation service can furthermore be configured to use a neural network in order to derive treatment schemes and/or medical diagnoses from the sensor data and/or the at least one medical data set. The calculation service can furthermore be configured to take into consideration comparable disease progressions of other patients, which it retrieves from a database, in the derivation of the treatment scheme. The processing load to be borne by the coordination unit can be kept low by the outsourcing to the calculation service. The calculation service is preferably provided by computers distributed in a communication network, thus a cloud system. According to other embodiments of the invention, the calculation service can be implemented by a server or a server farm of a hospital in which the coordination unit is used.

It is furthermore possible according to the invention that the treatment system has an assistance computer, which is coupled to the coordination unit via a data connection, wherein the assistance computer is configured to execute calculations for the coordination unit and/or to provide medical data sets and/or to provide sensor data to the coordination unit. The use of such an assistance computer is reasonable above all if the coordination unit is a portable device having restricted processing power, for example a tablet computer. Important programs and data can be kept ready in this case by the assistance computer and transferred as needed to the tablet computer. The assistance computer can take over carrying out extensive calculations according to the invention. The assistance computer can be, for example, a server of a hospital. The treatment system can be designed according to the invention so that it comprises a plurality of coordination units which use the assistance computer.

The assistance computer preferably has a database. According to one particularly advantageous variant of the invention, the database is an anesthesia database. The anesthesia database records items of information which were recorded by anesthesia devices and optionally further medical devices. The assistance computer can thus be used as an anesthesia server. However, other patient data or sensor data (for example dosing specifications for medications in dependence on the liver and kidney situation) can also be archived in the database. Furthermore, data on the availability of medical devices can be stored therein. This enables the availability of the medical devices to also be incorporated in the preparation of treatment schemes.

It is furthermore possible according to the invention that the assistance computer is configured to request items of information from a hospital information system via a data connection and to provide the items of information to the coordination unit, wherein the items of information comprise the sensor data and/or the medical data sets. A hospital information system is an EDP system, which assists greatly varying tasks from fields such as administration, documentation, and/or coordination within a hospital. For example, electronic patient files can be kept in the hospital information system. According to advantageous embodiments of the invention, it is possible that the coordination unit retrieves the content of patient files or other medical data sets from the hospital information system by means of the assistance computer. Diverse medical devices can be connected to the hospital information system and can supply sensor data thereto. It is therefore possible that the coordination unit requests sensor data from the hospital information system by means of the assistance computer. The hospital information system can be implemented on one or more computers of the hospital, however, it can also be embodied at least partially on a central server of the hospital or in a cloud system.

### BRIEF DESCRIPTION OF THE DRAWINGS

An advantageous embodiment of the invention is explained by way of example in the drawings. In the figures:

FIG. 1 shows a schematic illustration of a treatment system; and

FIG. 2 shows a flow chart having steps which are executed by a coordination unit to derive a treatment scheme.

### DETAILED DESCRIPTION OF EXEMPLARY EMBODIMENT

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

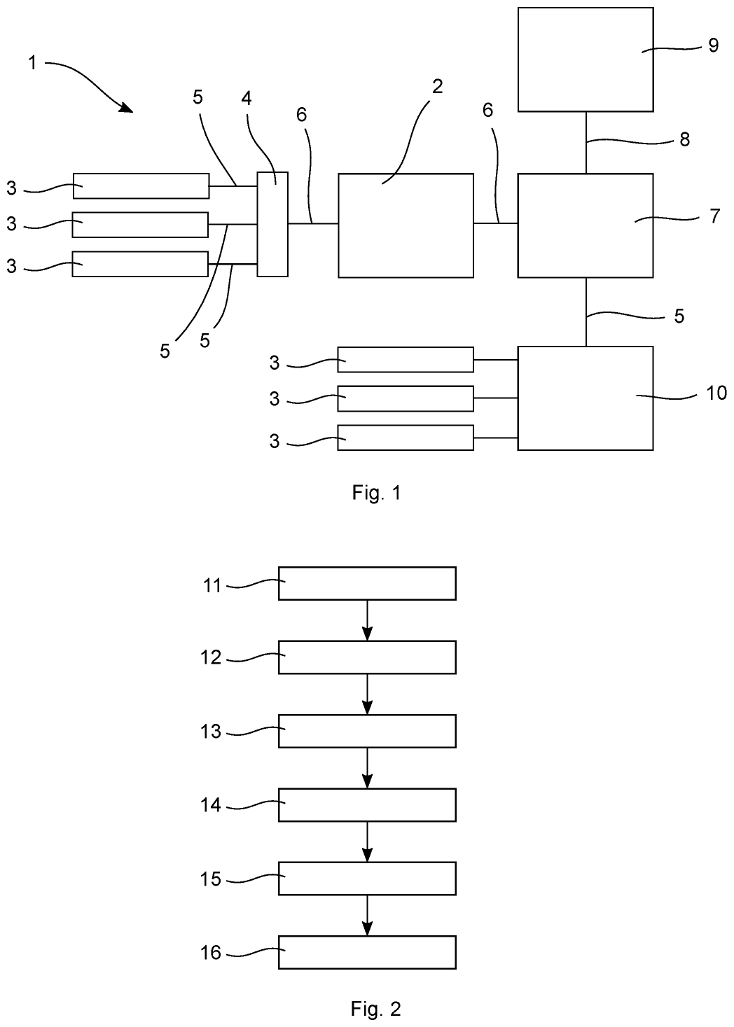

FIG. 1 shows a schematic illustration of a treatment system 1. The treatment system 1 has a coordination unit 2. The coordination unit 2 is connected to multiple medical devices 3 via a coupling unit 4. The coupling unit 4 is connected via wired data connections 5 to the medical devices 3. The coordination unit 2 is connected via a radio connection 6, which is a WLAN connection, to the coupling unit 4. The coordination unit 2 can thus receive sensor data from the medical devices 3 by means of the coupling unit 4 and also activate the medical devices 3.

The coordination unit 2 is connected via a further radio connection 6 to an assistance computer 7. The assistance computer 7 is used as an anesthesia server and permits the documentation of treatment steps during anesthesia. In addition, the assistance computer 7 can also store further treatment data, however. Furthermore, it provides treatment data and programs to the coordination unit 2. The assistance computer 7 can take over complex calculations which cannot be executed by the coordination unit 2. The assistance computer 7 is connected via an Internet connection 8 to a calculation service 9. The coordination unit 2 can thus send an inquiry to the calculation service 9 via the assistance computer 7. The inquiry can contain, for example, sensor data and a medical data set such as an excerpt from a patient file. The calculation service 9 is implemented by networked computers in a cloud system. The calculation service 9 provides services using methods from the field of artificial intelligence. It can thus derive treatment schemes, which contain medical diagnoses and action recommendations, from the sensor data and further medical data.

The assistance computer 7 is furthermore coupled to a hospital information system 10 via a further wired data connection 5. The hospital information system 10 assists tasks from the fields of administration, documentation, and coordination and is formed by a plurality of computers. The coordination unit 2 can request patient data from the hospital information system 10. Since at least some computers of the hospital information system 10 are also connected to medical devices 3, sensor values of the medical devices 3 can also be retrieved via this.

FIG. 2 shows a flow chart having steps which are executed by the coordination unit to derive a treatment scheme. In a first receiving step 11, the coordination unit receives sensor data. The sensor data originate from medical devices which are connected directly or indirectly to the coordination unit. In a second receiving step 12, the coordination unit receives a medical data set. The medical data set can originate, for example, from an electronic patient file. In a subsequent evaluation step 13, the coordination unit evaluates the sensor data and the medical data set. Processing steps required for this purpose can be executed directly by the coordination unit. In dependence on the processed stated object, processing steps can also be taken over by the assistance computer or by the calculation service, however. The result of the evaluation step 13 is a treatment scheme. The treatment scheme contains steps which are necessary for the medical treatment of a patient. In a first instruction step 14, the coordination unit activates at least one medical device so that the treatment scheme is implemented. In a second instruction step 15, the coordination unit outputs the treatment scheme on a display screen of the coordination unit, wherein instructions to medical personnel are contained in the treatment scheme. The coordination unit can thus also initiate treatment steps which cannot be implemented by the medical devices. In a diagnosis step 16, the coordination unit finally outputs a medical diagnosis which has been derived from the sensor data and the medical data set. The coordination unit thus assists the medical professionals in the diagnosis of diseases and can initiate the required steps for the treatment of patients.

To sum up, the instant invention provides the following items:

1. A coordination unit for a group of medical devices, wherein the coordination unit is configured to execute the following steps:
receiving sensor data from at least one of the medical devices, the sensor data being associated with a patient,
receiving a medical data set which is associated with the patient, and
initiating an evaluation of the sensor data and/or the at least one medical data set to derive a treatment scheme.

2. The coordination unit of item 1, wherein the coordination unit is furthermore configured to activate at least one of the medical devices so that an implementation of the treatment scheme takes place and/or implementation of the treatment scheme is assisted.

3. The coordination unit of item 2, wherein the coordination unit is configured to output the treatment scheme, to receive a confirmation signal, and, only upon receiving the confirmation signal, to activate the at least one of the medical devices so that the implementation of the treatment scheme takes place or the implementation of the treatment scheme is assisted, the treatment scheme including a medical diagnosis.

4. The coordination unit of any one of the preceding items, wherein the coordination unit is configured to initiate an output of a treatment scheme, and the coordination unit comprises a display screen to output the treatment scheme.

5. The coordination unit of any one of the preceding items, wherein the coordination unit comprises an input device in the form of touch display screen.

6. The coordination unit of any one of the preceding items, wherein the coordination unit is configured, to evaluate the sensor data and/or the at least one medical data set, to place a request to a calculation service and to receive a treatment scheme or data from which the coordination unit can derive the treatment scheme from the calculation service, and the coordination unit further is configured to place a database inquiry to a patient database.

7. The coordination unit of any one of the preceding items, wherein the coordination unit is configured to request an availability of the medical devices and, upon initiation of the evaluation of the sensor data and/or the at least one medical data set to derive a treatment scheme, to take into consideration the availability of the medical devices.

8. The coordination unit of any one of items 1 to 6, wherein the sensor data and/or the medical data set comprise measured values from a device for electrical impedance tomography, a device for blood gas analysis, and/or a ventilator, wherein the medical data set comprises at least one data value relating to a positioning of a patient, and wherein a treatment scheme derived from the sensor data and/or the at least one medical data set comprises an instruction for the positioning of the patient.

9. The coordination unit of any one of items 1 to 6, wherein the sensor data and/or the medical data set comprise at least one coagulation blood value and an oxygenation value, wherein a treatment scheme derived from the sensor data and optionally from the at least one medical data set comprises as a diagnosis a presence of microthrombi in a lung of a patient, and wherein the treatment scheme derived from the sensor data and/or the at least one medical data set provides an administration of an anticoagulant to the patient.

10. The coordination unit of item 9, wherein the sensor data furthermore comprise measured values from a device for electrical impedance tomography and/or a ventilator, and wherein the medical data set optionally comprises at least one data value on a positioning of the patient.

11. The coordination unit of any one of items 8 to 10, wherein the medical data set identifies that the patient is infected with a pathogen from the family of coronaviruses.

12. The coordination unit of any one of items 1 to 6, wherein the medical data set records that the patient suffers from muscle weakness acquired on an intensive care unit, wherein the sensor data and/or the medical data set comprise measured values from a device for electrical impedance tomography and/or from a ventilator, wherein the medical data set optionally indicates a sedation status of the patient, and wherein the treatment scheme derived from the sensor data and optionally from the at least one medical data set comprises at least one instruction specific for a withdrawal process from a ventilator.

13. The coordination unit of item 12, wherein the at least one instruction specific for the withdrawal process is a starting time for the withdrawal process, an ending time for the withdrawal process, or an instruction for adjusting a ventilation parameter.

14. The coordination unit of any one of items 12 or 13, wherein the treatment scheme comprises an instruction for setting a ventilation rate, for setting a level of a ventilation pressure, and/or for setting a tidal volume to enable spontaneous respiration.

15. The coordination unit of any one of items 1 to 6, wherein the sensor data originate from one or more of a device for electrical impedance tomography, a ventilator, a device for pulse oximetry, a hemoglobin meter, an esophageal pressure sensor, a device for blood pressure measurement, a computer tomography device, an x-ray device, an ultrasound device, a capnometer, an EEG device, or a device for blood gas analysis, and wherein a treatment scheme derived from the sensor data and/or the medical data set comprises at least one specific instruction for the treatment of an acute respiratory insufficiency of the patient.

16. The coordination unit of item 15, wherein the at least one specific instruction for the treatment of the acute respiratory insufficiency of the patient comprises one or more of an instruction for setting an inspirational O2 concentration, an instruction for setting an inspiration time, an instruction for setting a PEEP value, an instruction for setting a mandatory ventilation rate, an instruction for setting an individual trigger or an instruction for setting a sedation parameter.

17. A treatment system, wherein the treatment system comprises the coordination unit according to any one of items 1 to 16 and at least one medical device, the coordination unit being coupled to the medical device via a data connection.

18. The treatment system of item 17, wherein the treatment system comprises a calculation service, the coordination unit being coupled to the calculation service via a data connection, and the calculation service being formed by computers distributed in a communication network.

19. The treatment system of any one of items 17 and 18, wherein the treatment system comprises an assistance computer, which is coupled to the coordination unit via a data connection and comprises a database, the assistance computer being configured to execute calculations for the coordination unit and/or to provide medical data sets and/or to provide sensor data to the coordination unit.

20. The treatment system of item 19, wherein the assistance computer is configured to request items of information from a hospital information system via a data connection and provide the items of information to the coordination unit, the items of information comprising the sensor data and/or the medical data sets.

### LIST OF REFERENCE NUMERALS

1. treatment system

2. coordination unit

3. medical device

4. coupling unit

5. wired data connection

6. radio connection

7. assistance computer

8. Internet connection

9. calculation service

10. hospital information system

11. first receiving step

12. second receiving step

13. evaluation step

14. first instruction step

15. second instruction step

16. diagnosis step

What is claimed is:

1. A coordination unit for a group of medical devices, wherein the coordination unit is configured to execute the following steps:
receiving sensor data from at least one of the medical devices, the sensor data being associated with a patient,
receiving a medical data set which is associated with the patient, and
initiating an evaluation of the sensor data and/or the at least one medical data set to derive a treatment scheme.

2. The coordination unit of claim 1, wherein the coordination unit is furthermore configured to activate at least one of the medical devices so that an implementation of the treatment scheme takes place and/or implementation of the treatment scheme is assisted.

3. The coordination unit of claim 2, wherein the coordination unit is configured to output the treatment scheme, to receive a confirmation signal, and, only upon receiving the confirmation signal, to activate the at least one of the medical devices so that the implementation of the treatment scheme takes place or the implementation of the treatment scheme is assisted, the treatment scheme including a medical diagnosis.

4. The coordination unit of claim 1, wherein the coordination unit is configured to initiate an output of the treatment scheme and comprises a display screen to output the treatment scheme.

5. The coordination unit of claim 1, wherein the coordination unit comprises an input device in the form of touch display screen.

6. The coordination unit of claim 1, wherein the coordination unit is configured, to evaluate the sensor data and/or the at least one medical data set, to place a request to a calculation service and to receive a treatment scheme or data from which the coordination unit can derive the treatment scheme from the calculation service, and the coordination unit further is configured to place a database inquiry to a patient database.

7. The coordination unit of claim 1, wherein the coordination unit is configured to request an availability of the medical devices and, upon initiation of the evaluation of the sensor data and/or the at least one medical data set to derive a treatment scheme, to take into consideration the availability of the medical devices.

8. The coordination unit of claim 1, wherein the sensor data and/or the medical data set comprise measured values from one or more of a device for electrical impedance tomography, a device for blood gas analysis, or a ventilator, wherein the medical data set comprises at least one data value relating to a positioning of a patient, and wherein the treatment scheme derived from the sensor data and/or the at least one medical data set comprises an instruction for the positioning of the patient.

9. The coordination unit of claim 1, wherein the sensor data and/or the medical data set comprise at least one coagulation blood value and an oxygenation value, wherein the treatment scheme derived from the sensor data and/or the at least one medical data set comprises as a diagnosis a presence of microthrombi in a lung of a patient, and wherein the treatment scheme derived from the sensor data and/or the at least one medical data set provides an administration of an anticoagulant to the patient.

10. The coordination unit of claim 9, wherein the sensor data furthermore comprise measured values from a device for electrical impedance tomography and/or a ventilator, and wherein the medical data set optionally comprises at least one data value on a positioning of the patient.

11. The coordination unit of claim 8, wherein the medical data set identifies that the patient is infected with a pathogen from the family of coronaviruses.

12. The coordination unit of claim 1, wherein the medical data set records that the patient suffers from muscle weakness acquired on an intensive care unit, wherein the sensor data and/or the medical data set comprise measured values from a device for electrical impedance tomography and/or from a ventilator, wherein the medical data set optionally indicates a sedation status of the patient, and wherein a treatment scheme derived from the sensor data and/or the at least one medical data set comprises at least one instruction specific for a withdrawal process from a ventilator.

13. The coordination unit of claim 12, wherein the at least one instruction specific for the withdrawal process is a starting time for the withdrawal process, an ending time for the withdrawal process, or an instruction for adjusting a ventilation parameter.

14. The coordination unit of claim 12, wherein the treatment scheme comprises an instruction for setting a ventilation rate, for setting a level of a ventilation pressure, and/or for setting a tidal volume to enable spontaneous respiration.

15. The coordination unit of any claim 1, wherein the sensor data originate from one or more of a device for electrical impedance tomography, a ventilator, a device for pulse oximetry, a hemoglobin meter, an esophageal pressure sensor, a device for blood pressure measurement, a computer tomography device, an x-ray device, an ultrasound device, a capnometer, an EEG device, or a device for blood gas analysis, and wherein a treatment scheme derived from the sensor data and/or the medical data set comprises at least one specific instruction for a treatment of an acute respiratory insufficiency of the patient.

16. The coordination unit of claim 15, wherein the at least one specific instruction for the treatment of the acute respiratory insufficiency of the patient comprises one or more of an instruction for setting an inspirational O2 concentration, an instruction for setting an inspiration time, an instruction for setting a PEEP value, an instruction for setting a mandatory ventilation rate, an instruction for setting an individual trigger or an instruction for setting a sedation parameter.

17. A treatment system, wherein the treatment system comprises the coordination unit of claim 1 and at least one medical device, the coordination unit being coupled to the medical device via a data connection.

18. The treatment system of claim 17, wherein the treatment system comprises a calculation service, the coordination unit being coupled to the calculation service via a data connection, and the calculation service being formed by computers distributed in a communication network.

19. The treatment system of claim 17, wherein the treatment system comprises an assistance computer, which is coupled to the coordination unit via a data connection and comprises a database, the assistance computer being configured to execute calculations for the coordination unit and/or to provide medical data sets and/or to provide sensor data to the coordination unit.

20. The treatment system of claim 19, wherein the assistance computer is configured to request items of information from a hospital information system via a data connection and provide retrieved items of information to the coordination unit, the items of information comprising the sensor data and/or the medical data sets.
